(19) United States
(12) Patent Application Publication
(10) Pub. No.: US 2021/0346013 A1
(43) Pub. Date: Nov. 11, 2021

(54) SINGLE ARM PERFORMING AUTOSTITCHING DEVICE

(71) Applicant: Children's Medical Center Corporation, Boston, MA (US)

(72) Inventor: Kaifeng Liu, Boston, MA (US)

(21) Appl. No.: 17/313,087

(22) Filed: May 6, 2021

Related U.S. Application Data
(60) Provisional application No. 63/021,991, filed on May 8, 2020.

(51) Int. Cl. A61B 17/062

(52) U.S. Cl. CPC A61B 17/0625; A61B 2017/00876

(57) ABSTRACT

A suturing device is provided that includes a handle with a rod extending therefrom and a needle member into which the rod is inserted to engage therewith. A suture is attached to an outer surface of the needle member. The needle member includes expandable fins formed at a proximate end thereof that expand after the needle member is passed through a tissue to prevent a backward movement of the needle member in a suturing procedure.

### CROSS REFERENCE TO RELATED APPLICATION

The present application claims priority to a U.S. provisional application 63/021,991, filed May 8, 2020, the entire contents of which are incorporated herein for all purposes by this reference.

### BACKGROUND

### 1. Technical Field

The present disclosure relates generally to a suturing apparatus and more particularly, to a suturing apparatus in which a needle is automatically loaded onto a holder of the apparatus to simplify the suturing process.

### 2. Description of the Related Art

In general, stitching or suturing, in a medical field, is used to bind pieces of material together. Specifically, suturing is used to stitch together tissue during a surgical procedure. The pieces of tissue will then fuse together during a healing process. A suture needle is typically used to force a suture thread through the layers of tissue to allow the thread to bind the tissue layers together. This suturing process is both time consuming and requires a practitioner to grasp multiple components simultaneously with high accuracy.

A typical suture needles includes a needle tip at one end and a suture connection point at the other end. The length of the suture needle also depends on the type of procedure being performed. For example, a longer suture needle is used for large wounds while a short suture needle is used when access or room for manipulation is limited, such as in small and deep spaces. The tip of the needle is inserted through a material or tissue by applying insertion force to the suture needle. Additional force is then used to guide the remainder of the suture needle through the material until the entire needle is passed therethrough. Since the suture thread is coupled to the suture connection point, the thread is pulled along when the insertion force is applied to the suture needle. The process of inserting the needle with the suture thread through the material or tissue is repeated with another layer of material to bind the materials together.

Developed surgical tools for assisting in this suturing process include instruments that clamp the end of the needle to more easily apply force to the needle through the tissue. In particular, this type of tool requires a user to maintain the clamp grasped against the needle while in use. The clamp is then released once the needle has penetrated through the material or tissue. This instrument, however, requires particular user positioning in order to grasp the needle and also requires continuous manipulation of the clamp. When the needle is to be inserted in a small space, as discussed above, such positioning may be difficult. Additionally, during the operation of the clamp, the needle is unable to be continuously maintained in a secure position, thus increasing potential contamination risk if dropped from the clamp.

According to another developed design, a pair of tweezers may include the suture needle between the tips thereof. In particular, a straight double-ended suture needle is moveable between the tips of the tweezers upon engagement of the tweezers. For example, when a user squeezes the tweezer arms together, the suture needle disconnects from one tip and engages with the other tip. The engagement is based on the release of a blade inside the tweezer arm which must capture a groove of the needle and hold the needle within a recess of the arm. When the tweezers are engaged, the other end of the needle enters an opposite recess and another blade must precisely engage with the needle groove to lock the needle in place. The design, however, is complex requiring multiple interworking elements within the tweezer arms to lock the needle end in place, thus increasing risk of error use.

According to yet another developed design, a suture capturing device has been developed that utilizes a funnel suture dart catch design and a 3-finger grip. For operation, a trigger is squeezed to provide tension along a wire which pulls back a needle within the capturing device. For example the trigger is squeezed and released to move the needle between an engaging and disengaging position. However, such a device requires consistent tension along a wire that holds the position of a needle at the suture site. This increases the risk of inadvertent release of the needle where undesirable. On the other hand, if excessive tension is exerted, the needle may bend or be otherwise deformed causing damage to the overall system.

### SUMMARY

The present disclosure provides a single arm auto-stitching device and method thereof in which a needle member is attracted onto a holder of the device to simplify a suturing process, thus decreasing the required time for suturing, and provides a more stable and continuous grip onto the needle while puncturing through a material without requiring excessive tension to be held during such a process.

According to one aspect of the present disclosure, a suturing device may include a handle with a rod extending therefrom and a needle member into which the rod may be inserted to engage therewith. Additionally, a suture may be attached to an outer surface of the needle member. The needle member may include expandable fins formed at a proximate end thereof. The needle member may be hollow with a pointed tip at a distal end thereof to pierce through a tissue layer.

According to another exemplary embodiment, the needle member may be a cylindrical hollow member with both ends thereof open. The rod extending from the handle may have a pointed tip that extends through the needle member when engaged therewith.

Further, an interior of the needle maybe magnetic to be engaged with a magnetic end of the rod. Additionally, the fins of the needle member may expand to a diameter greater than an outer diameter of the needle member. The fins may also retract to be in line with the outer surface of the needle member. In particular, the fins may expand after being inserted through the tissue layer to prevent backward movement of the needle member into the tissue layer.

According to another exemplary embodiment, the needle member may include at least one protrusion formed along a circumferential surface thereof. Additionally, the needle member may be expandable within a tissue layer. A plurality of rods may extend from the handle and a plurality of needle members may be provided to receive each of the rods.

According to another aspect of the present disclosure, a suturing method is provided. The method may include engaging a rod that extends from a handle of a suturing device into a needle member. A suture may be attached to an outer surface of the needle member. The method may then include piercing a tip of the needle member through a tissue layer. Fins may be formed at a proximate end of the needle member and may expand after passing through the tissue member.

Further, as the needle member passes through the tissue layer, the fins may be retracted to be in line with the outer surface of the needle member. The fins may expand to a diameter greater than an outer diameter of the needle member to prevent backward movement into the tissue layer.

The method may further include disengaging the rod from the needle member once the fins are expanded. The rod may then be reengaged into the needle member to pass the needle member through another tissue layer.

Notably, the present invention is not limited to the combination of the suturing apparatus element as listed above and may be assembled in any combination of the elements as described herein.

Other aspects of the disclosure are disclosed infra.

### BRIEF DESCRIPTION OF THE DRAWINGS

The embodiments herein may be better understood by referring to the following description in conjunction with the accompanying drawings in which like reference numerals indicate identically or functionally similar elements, of which:

FIGS. 1A-1B illustrate a suturing device according to an exemplary embodiment of the present disclosure;

FIGS. 2A-2B illustrate an engagement of a rod and needle member of the suturing device according to an exemplary embodiment of the present disclosure;

FIGS. 3A-3B illustrate a detailed view of the needle member according to an exemplary embodiment of the present disclosure;

FIGS. 4A-4B illustrate a detailed view of the handle and rod according to an exemplary embodiment of the present disclosure;

FIGS. 5A-5L illustrate a method of operating a suturing device according to an exemplary embodiment of the present disclosure;

FIGS. 6A-6D illustrate a suturing device according to another exemplary embodiment of the present disclosure;

FIGS. 7A-7B illustrate a suturing device according to another exemplary embodiment of the present disclosure;

FIG. 8 illustrates a detailed view of the suture according to another exemplary embodiment of the present disclosure;

FIGS. 9A-9G illustrate the needle member according to an exemplary embodiment of the present disclosure; and

FIG. 10 illustrates an expandable suture according to an exemplary embodiment of the present disclosure.

It should be understood that the appended drawings are not necessarily to scale, presenting a somewhat simplified representation of various features illustrative of the basic principles of the disclosure. The specific design features of the present disclosure as disclosed herein, including, for example, specific dimensions, orientations, locations, and shapes will be determined in part by the particular intended application and use environment.

### DETAILED DESCRIPTION

The presently disclosed subject matter will be described more fully hereinafter with reference to the accompanying drawings, in which some, but not all embodiments of the inventions are shown. The presently disclosed subject matter may be embodied in many different forms and should not be construed as limited to the embodiments set forth herein; rather these exemplary embodiments are provided so that this disclosure will satisfy applicable legal requirements. Indeed, many modifications and other exemplary embodiments of the presently disclosed subject matter set forth herein will come to mind to one skilled in the art to which the presently disclosed subject matter pertains, having the benefit of the teachings presented in the foregoing descriptions and the associated drawings. Therefore, it is to be understood that the presently disclosed subject matter is not limited to the specific embodiments disclosed and that modifications and other exemplary embodiments are intended to be included within the scope of the appended claims.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

In one aspect, the present disclosure provides a suturing system that is capable of performing auto-stitching using a single-armed device. That is, a needle member with a suture attached thereto is capable of engaging with a rod of the device to puncture through tissue while preventing any backward movement of the needle member into the tissue due to fins extending from an end thereof. In other words, when the rod engaged with the needle member is pulled out from the tissue, there is no risk of the needle following the rod as the fins operate as a stopper for the needle member. Additionally, the device is capable of stably holding the needle in place while puncturing through the tissue, simplifying the suturing process, decreasing suturing time, and increasing suturing accuracy. A user is no longer required to grasp a small needle while pushing the needle through a tissue. Instead, the needle is auto-loaded onto the rod and is held in an engagement with the rod while the needle is pushed through the tissue. This also allows a user to place stitches in typically inaccessible locations.

In surgical applications, the suture of the apparatus may be a suture thread designed for use with organic tissue and may be composed of an absorbable or non-absorbable material. For example, the thread may be composed of an absorbable material such as catgut, polyglycolic acid, polyactic acid, polydioxanone, caprolactone, or the like. Exemplary non-absorbable materials include polypropylene, polyester, nylon, metallic wires, and the like. In some cases, the thread may be coated with a compound that reduces friction during the suturing process, has antibacterial properties, and/or produces a biological reaction in the patient/subject (e.g., acts as an anti-inflammatory, etc.).

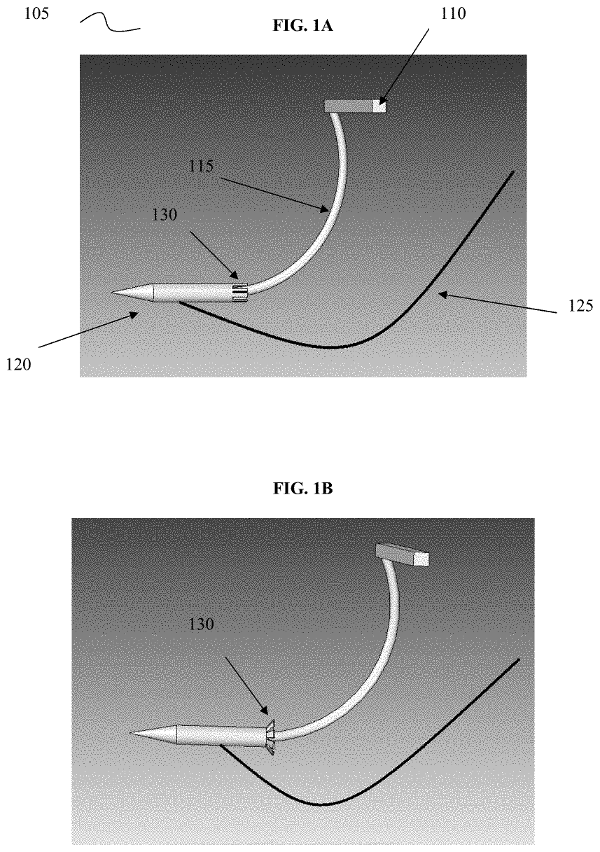

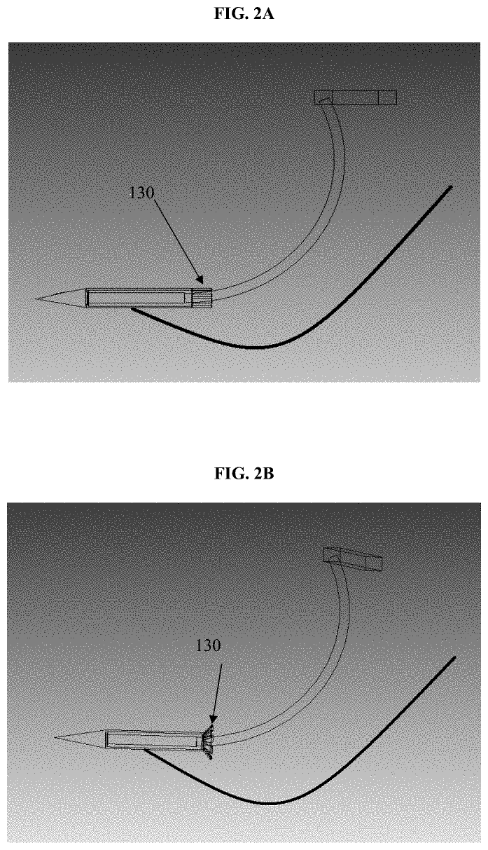

Referring now to FIGS. 1A-1B and 2A-2B, a suturing device 105 is shown according to an exemplary embodiment of the present disclosure. In particular, the suturing device 105 may include a handle 110 with a rod 115 extending from the handle. A needle member 120 is provided into which the rod 115 may be inserted to engage therewith. Additionally, a suture 125 may be attached to an outer surface of the needle member 120. The needle member 120 may further include expandable fins 130 formed at a proximate end of the needle member 120.

In particular, the needle member 120 may be formed to be hollow with a pointed tip at a distal end thereof used to pierce through a tissue layer or other material. Notably, the needle member 120 is not limited to such a shape and other examples thereof will be described herein below. The interior of the needle member 120 (shown in FIGS. 2A-2B) may be magnetic to be engaged with a magnetic end of the rod 115 (e.g., the pointed tip thereof). Accordingly, when the rod 115 approaches or comes into close contact with the needle member 120, the two components are attracted to each other due to the magnetic force therebetween and to engage with each other, the rod 115 is inserted or attracted into the opening of the needle member 120.

Notably, the figures illustrate that the rod 115 extends in a curved shape from the handle 110, however the present disclosure is not limited thereto. The rod 115 may extend from the handle 110 at variable angles. This facilitates access to, for example, surgical regions provided at difficult to reach locations.

The magnetic engagement between the needle member 120 with the suture 125 attached thereto and the rod 115 facilitates the ability to operate the device in typically unreachable or limited spatial regions. In other words, a user is not required to physically reposition to reach a surgical region and perform the suturing process since the rod 115 and needle member 120 are automatically engaged and re-engaged with each other during a suturing process. A further description of such a process will be discussed herein below. Notably, the attraction between the magnetic needle member 120 and the magnetic tip of the rod 115 may be based on a permanent magnet, electromagnet, or a combination thereof with the magnetic strength and direction being adjustable.

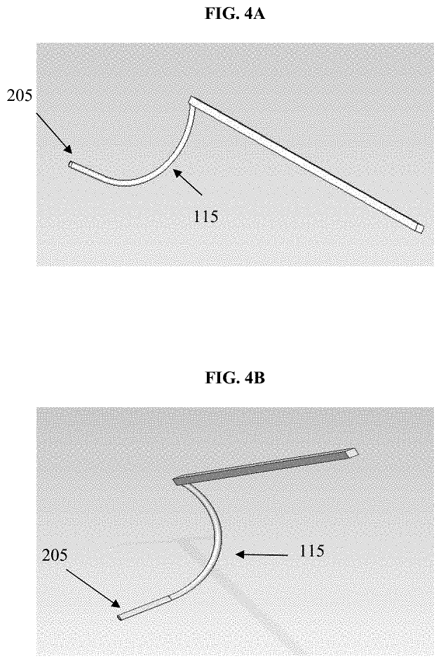

As shown in FIGS. 2A-2B and FIGS. 4A-4B, once the end 205 of the rod 115 is inserted into the needle member 120, the inner wall of the needle member prevents movement of the rod therein to provide stable engagement between the components. That is, the inner diameter of the needle member may be minimally greater than the outer diameter of the rod to thus restrict the movement of the rod once inserted into the needle member. This advantageously provides a stable engagement between the components while puncturing tissue without requiring the user to graph the needle member and also improves accuracy of tissue puncturing (e.g. prevents needle wobbling).

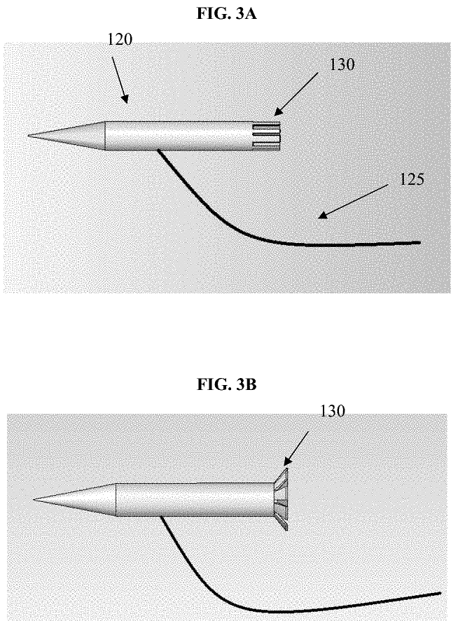

Furthermore, as shown in FIGS. 3A-3B, the fins 130 extending from a proximate end of the needle member 120 may be expandable (FIG. 3B) and retractable (FIG. 3A) based on a movement direction. In particular, as will be described in further detail below, the fins 130 may be formed in an expanded or flared shape with respect to an outer surface of the needle member 120. That is, the fins 130 may be flared to a greater diameter than the outer diameter of the needle member 120. Notably, the fin shape may be varied in both size and length. This may be referred to as an original position of the fins 130. When the needle member 120 punctures the tissue layer and is passed therethrough, the fins 130 may be flexible and retract to be in line with the outer surface of the needle member. Once the fins 130 have passed through the tissue layer, the fins 130 may expand back to the original position. This flaring of the fins 130 prevents the needle member 120 from moving or sliding back through the tissue, even partially, as the rod is extracted from the tissue to perform a suturing process.

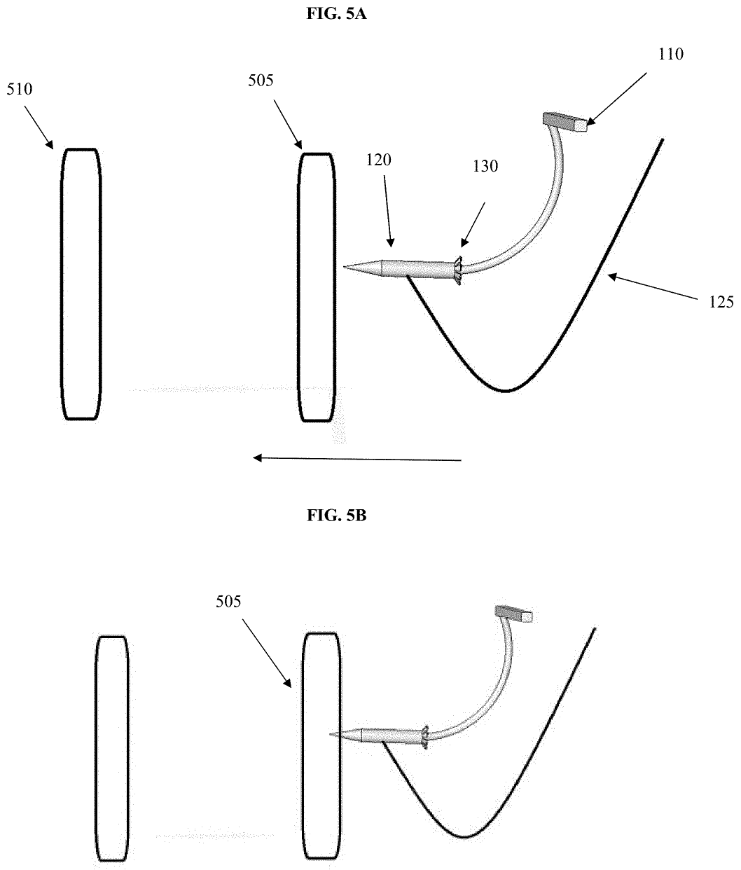

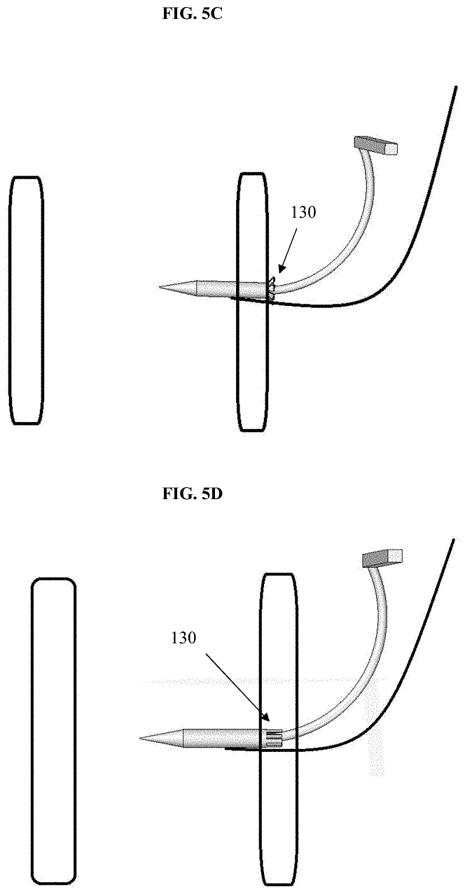

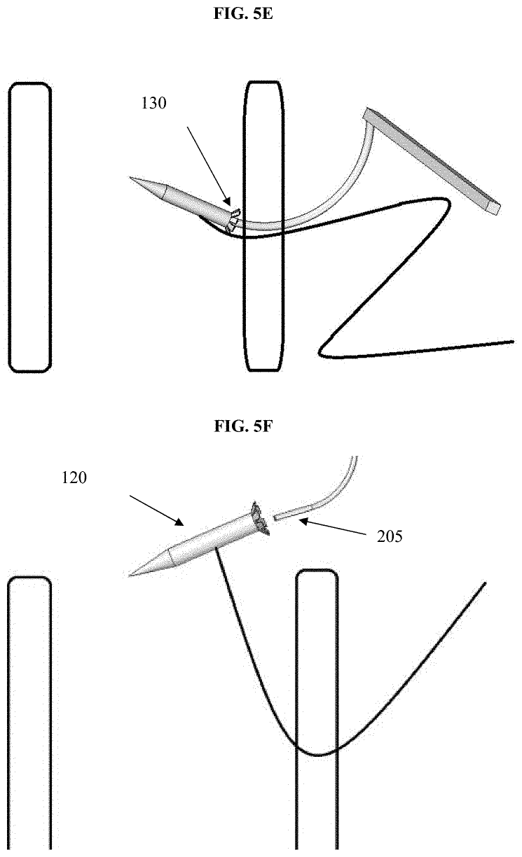

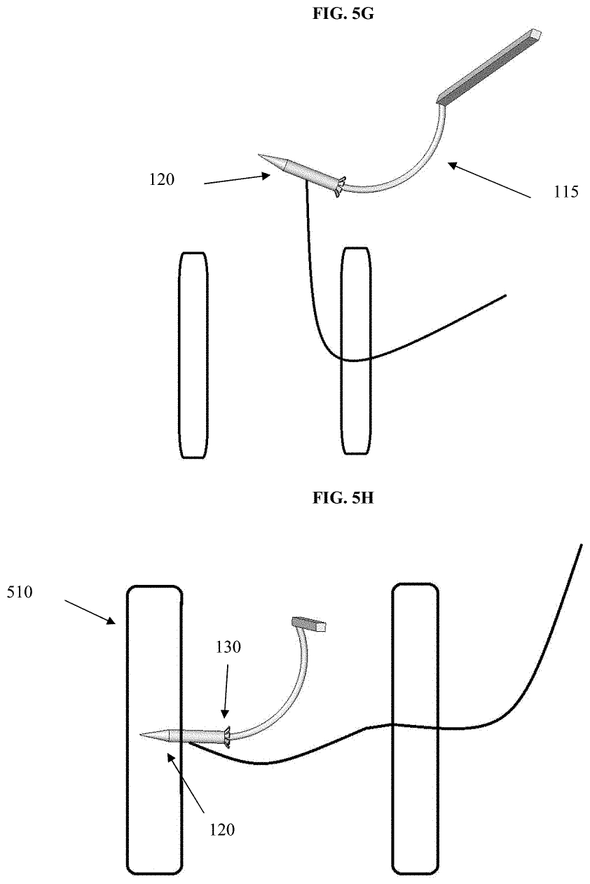

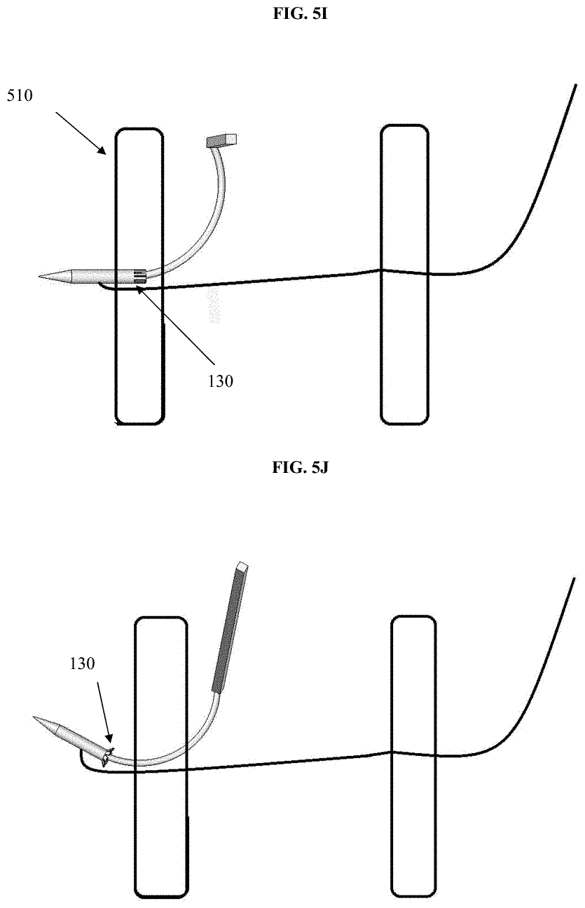

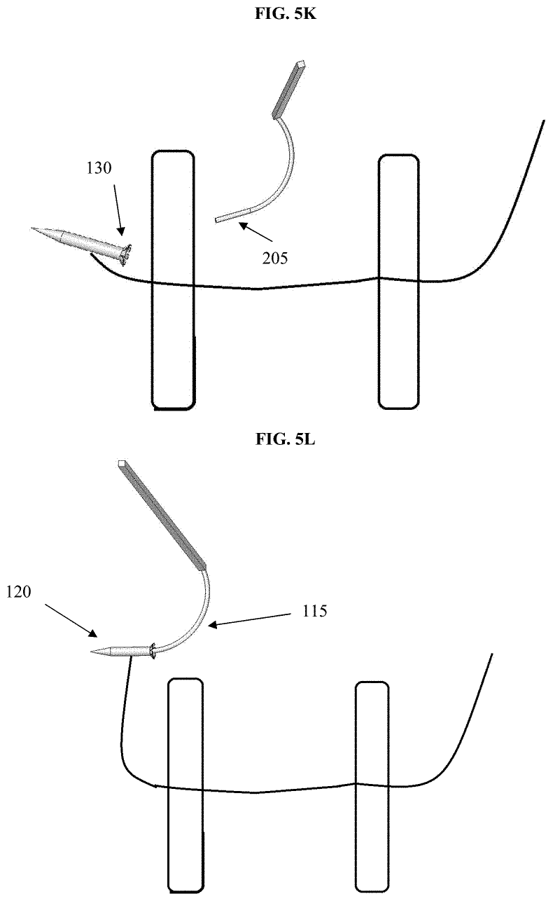

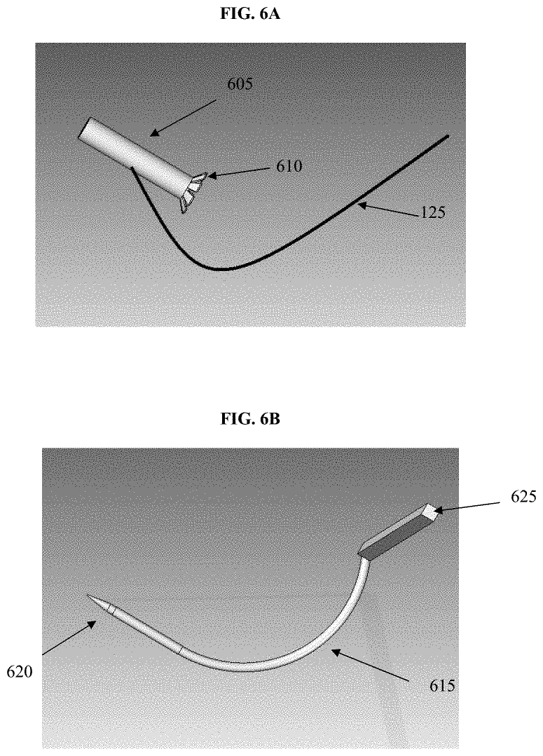

A process of performing suturing using the suturing device of the present disclosure will now be described with references to FIGS. 5A-5L. As described above and as shown in FIG. 5A, a suture 125 may be attached to an outer surface of the needle member 120 and a magnetic tip 205 of the rod 115 may be inserted into and engaged with the magnetic interior of the needle member 120. The arrow shown in FIG. 5A illustrates the moving direction of the suturing device 105. As illustrated in FIG. 5B, the suturing device 105 may be moved to puncture a first tissue layer 505. As shown, in this state, the fins 130 of the needle member 120 are in an original position, that is, flared beyond the outer diameter of the needle member 120.

FIG. 5C shows that the suture 125 moves through the first tissue layer 505 together with the needle member 120 and FIG. 5D shows the retraction of the fins 130 as the fins 130 pass through the first tissue layer 505. Further, as shown in FIG. 5E, once the entire needle member 120 including the fins 130 passes through the first tissue layer 505, the fins expand back to the original position (e.g., flared). This immediate flaring of the fins prevents the needle member from sliding back into the punctured first tissue layer. That is, the rod 115 is able to be extracted or pulled out of the tissue layer 505 and during such an extraction, the fins 130 press against the punctured tissue opening holding the needle member in place while allowing disengagement of the rod 115 from the needle member 120. By the fins 130 pressing against the tissue, no separate tool or maneuver is required for releasing or separating the rod 115 and the needle member 120.

As shown in FIGS. 5F and 5E, the rod 115 may then re-engage with the needle member 120 due to the magnetic attraction therebetween. That is, the rod 115 may be moved towards the opening of the needle member to be reinserted therein. Once re-engaged, FIG. 5H illustrates that the suturing device 105 may pierce through the second tissue layer 510 to continue the suturing process. Again, as shown in FIG. 5H, the fins 130 remain in the original position, or flared position, and then as shown in FIG. SI, retract again while passing through the second tissue layer 510. FIG. 5J illustrates the entire needle member 120 being passed through the second tissue 510 at which point the fins 130 are flared again. As shown, the rod remains in the puncturing state of the second tissue layer 510. FIG. 5K illustrates the disengagement of the rod 115 from the needle member 120. That is, the rod 115 may be extracted back out the second tissue layer 510 and release from the needle member 120 due to the fins 130 preventing the needle member from sliding back into the punctured tissue layer. As shown in FIG. 5L, the components may then continue to be re-engaged to complete a suturing process.

This process allows a user to perform the suturing process single handedly without requiring additional tools. The overall suturing process is thus simplified and also requires less spatial access which increases the possibility of suturing in limited space regions.

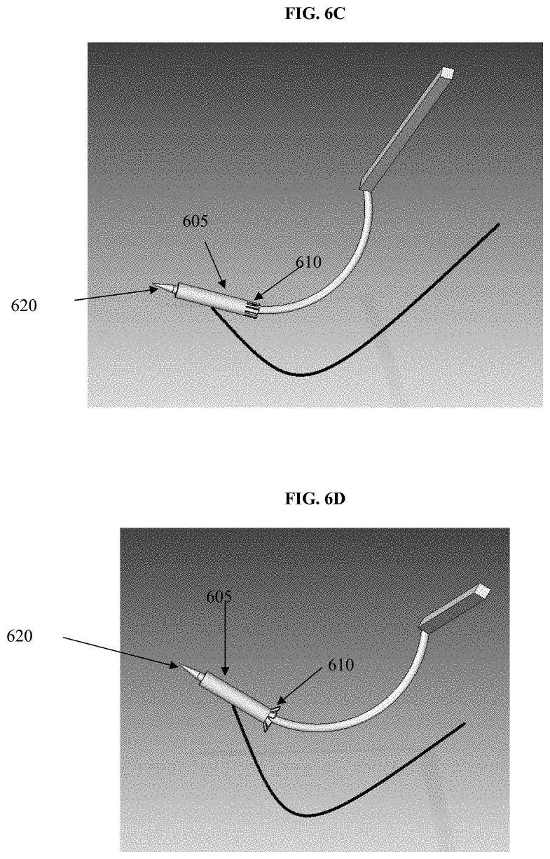

Moreover, as described briefly above, the needle member and road are not limited to the configurations as described above. For example, as shown in FIGS. 6A-6D, according to another exemplary embodiment of the present disclosure, the needle member 605 may be a cylindrical hollow member with both ends thereof open. The fins 610 in this configuration may still extend from the proximate end of the needle member 605. Additionally, as shown in FIG. 6B, the rod 615 extending from the handle 625 may have a pointed tip 620 to function as the needle tip that pierces through the tissue layer. That is, in this configuration, the rod itself may operate as a needle tip to pierce through the tissue layers. The tip 620 of the rod 615 may extend through the needle member when engaged therewith as shown in FIGS. 6C and 6D. In this configuration, the interior of the needle member may be magnetically engaged with a magnetic surface of the rod. For example, the entire outer surface of the rod or a portion of the outer surface of the rod may be magnetic (e.g., areas other than the tip).

In this configuration, the needle member 605 may be pressed fitted onto the rod 615 to provide sufficient tension to prevent the needle member 605 from sliding down along the rod 615 as the pointed tip 620 of the rod 615 pierces through the tissue layer. In other words, the diameter of the rod 615 may gradually increase from the tip 620 toward the 625 to prevent the needle member 605 from sliding towards the handle 625 when engaged with the rod 615. This press fit engagement may also be increased or tightened as the needle member and rod pass through the tissue layer.

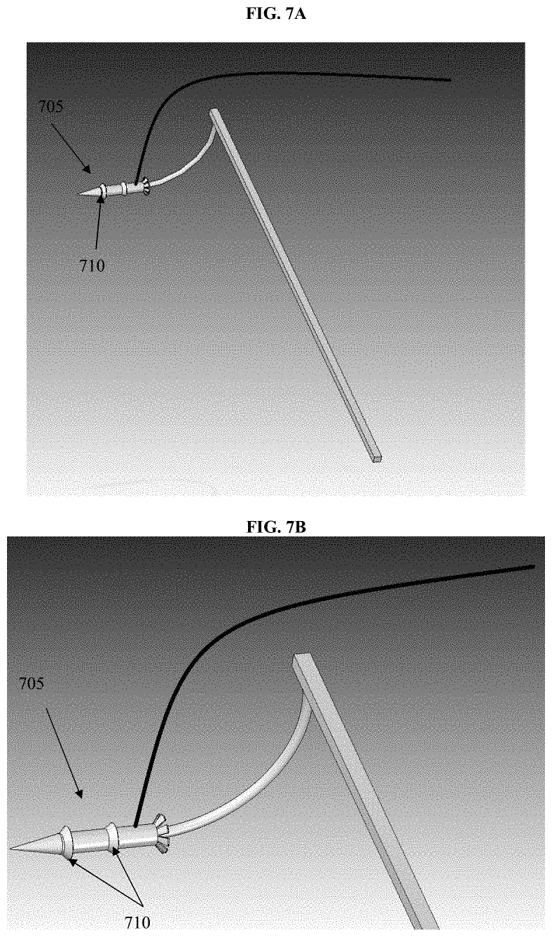

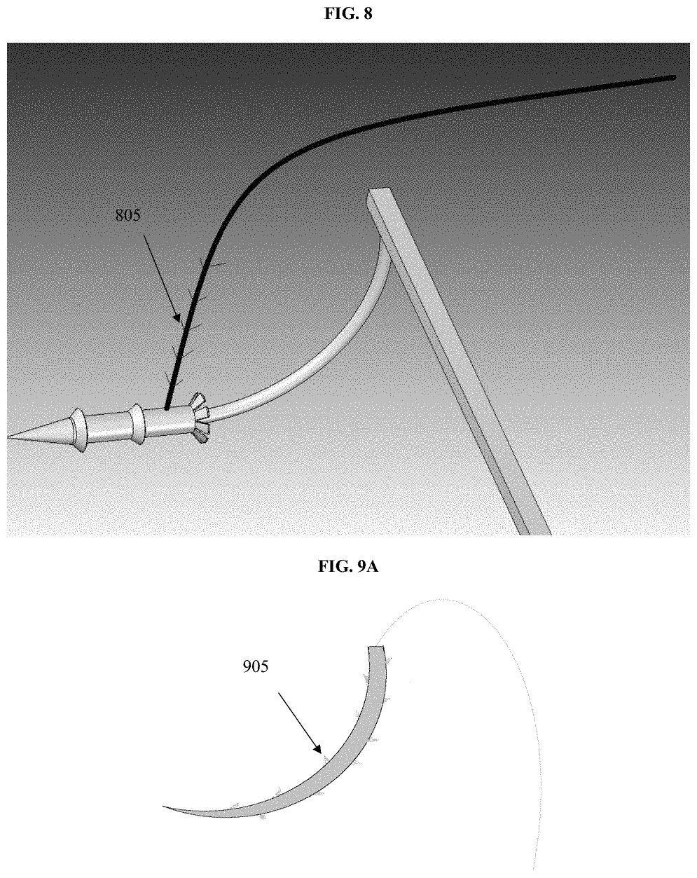

According to another exemplary embodiment of the present disclosure, as shown in FIGS. 7A-7B, the needle member 705 may be formed as the needle member 120 in FIGS. 1A-1B. In addition, the needle member 705 may further include at least one protrusion 710 formed along a circumferential surface thereof. Such a protrusion may be considered as an additional wing along the needle member. The wing or protrusion 710 may be formed integrally with the needle member. Additionally, the protrusion 710 may be formed of a metal, nylon, or similar material. For example, when a needle member is formed with merely a protrusion (e.g., without retractable fins), the protrusion may be formed at a steep angle providing resistance that prevents the needle member from sliding back through the tissue layer once punctured therethrough. Additionally, as shown in FIG. 8, the suture 805 may include wings 810 protruding therefrom to provide further friction for preventing backward movement through the tissue layer.

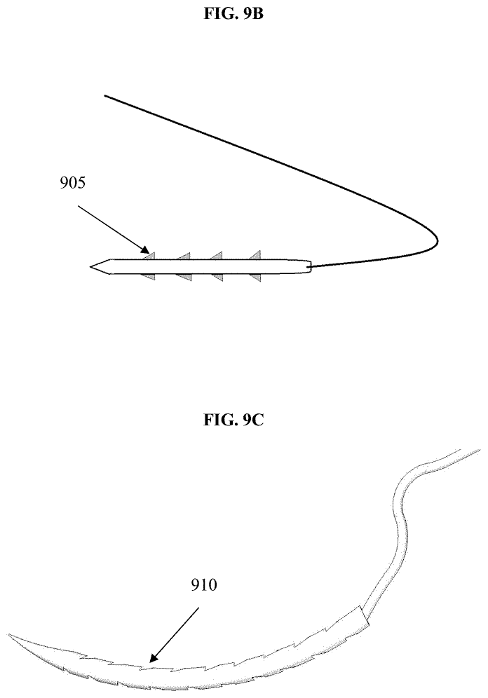

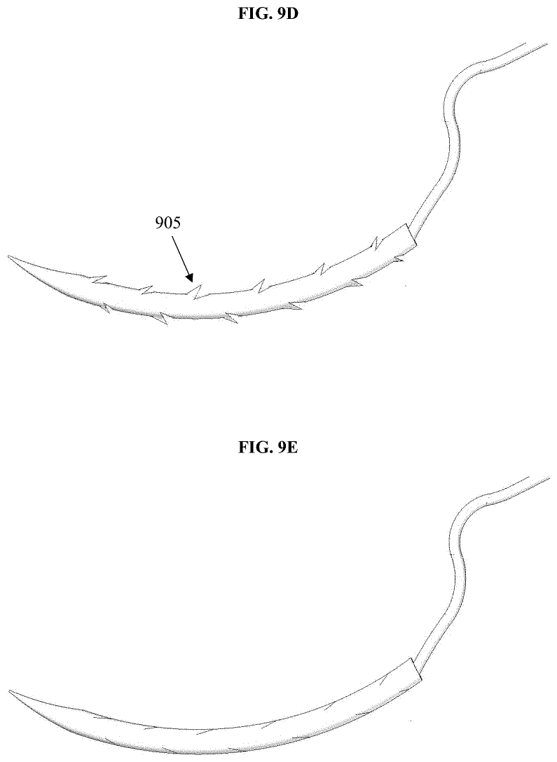

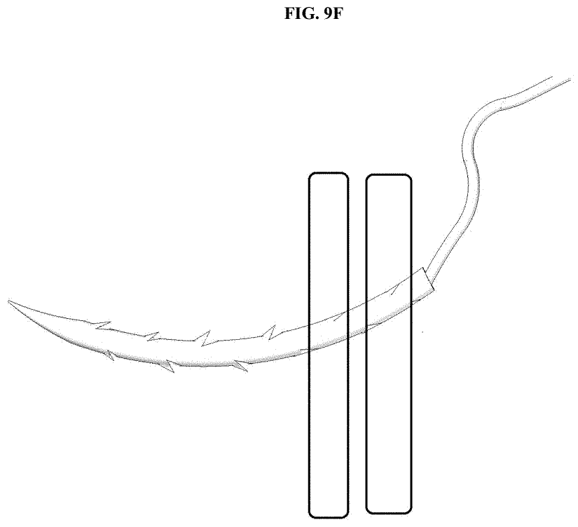

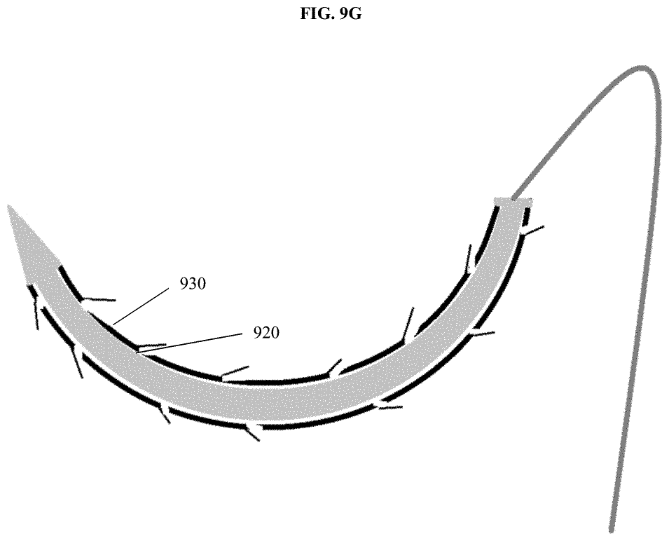

Referring to FIGS. 9A-9B, the surface of the needle member may include a plurality of protrusions 905 (e.g., thorns or wings). Additionally, FIGS. 9C-9F further show a groove 910 formed in the needle surface from the protrusions 905. These types of protrusions provide increased resistance when the needle member is extracted from the tissue. In other words, these protrusions provide smooth entry in the tissue layer but when extracted from the tissue layer in an opposite direction, provide increased resistance due to the configuration thereof. Alternately, these protrusions may be formed in line with the outer diameter of the needle member (see FIG. 9E) while advancing through the tissue and may flare when being pulled in a reverse direction (see FIG. 9F).

The protrusions may be formed in varying lengths and also in varying depths along the entire length of the needle member or merely a portion thereof. The spacing between each protrusion may also be the same or varying. For example, the length of the protrusions may increase in a direction towards the tip of the needle. In addition, as shown in FIG. 9G, the needle member may be formed in multiple layers. For example, an inner layer 920 of the needle member may be formed of a metal material, such as, stainless steel. An outer layer 930 of the needle member may be formed of a nylon, silicon, prolene, or similar material. In other words, the inner layer may be wrapped with a nylon or similar material to form an outer layer. The protrusions may be formed as slits in the outer layer of the needle member and thus, when the protrusions flare, the inner layer may be exposed. The outer layer may be resilient to prevent the needle member from passing back through the tissue layer once the protrusion are flared.

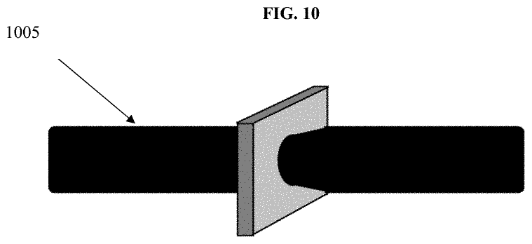

According to another exemplary embodiment, as shown in FIG. 10, the suture 1005 may be formed as an expandable suture. That is, the suture may be formed to expand at the puncture site of the needle to thus act as a stopper and anchor the tissue. Further, the needle shape itself may be formed in a variety of shapes. For example, the needle member may be formed to be straight, curved, in a coil shape, a screw shape, or a combination thereof. Additionally, a plurality of needle members may be provided. In other words, the handle of the suturing device may include a plurality of rods extending therefrom, each engaging with a needle member. This configuration allows multiple tissues sites to be punctured or pierced simultaneously thus improving the time required for completing a suturing process. Additionally, the handle may be formed to have rods magnetically engaged therewith. That is, the handle may be formed with one rod integrally formed therewith and may have a magnetic surface to which additional rods may engage to thus interact with a plurality of needle members. Accordingly, any selectable number of needle members may engage with the handle of the suturing device.

Notably, the engagement of the needle member and the rod is not limited to the above-described magnetic connection. The engagement may be via a variety of attractive forces such as mechanical, magnetic, and adhesive techniques that are capable of adjusting the magnetic field strength and direction. Alternatively, the suturing system may include power electronics that provide current to the windings of an electromagnet from a power supply via wires, to induce a magnetic field. Power electronics and/or power supply may be housed within the handle of the system. For example, the power supply may include one or more batteries that allow the system to be fully portable. In another example, the power supply may be external to the system. The power electronics operate to control the flow of current to the electromagnet in one or more directions.

The suturing device according to the present disclosure provides numerous advantages. For example, as discussed herein, the time required to perform suturing may be decreased based on the simplified system and the ability to operate the system with one hand. The system facilitates suturing in traditionally less accessible locations while also providing for more accurate suturing. Additionally, due to the strong engagement between the rod of the suturing device and the needle member while puncturing a material, the risk of inadvertently puncturing neighboring tissue or dropping the needle is substantially decreased. Notably, the present disclosure is not limited to surgical applications and may be used in any other application requiring connection of materials.

The many features and advantages of the disclosure are apparent from the detailed specification, and thus, it is intended by the appended claims to cover all such features and advantages of the invention which fall within the true spirit and scope of the disclosure. Further, since numerous modifications and variations will readily occur to those skilled in the art, it is not desired to limit the invention to the exact construction and operation illustrated and described, and accordingly, all suitable modifications and equivalents may be resorted to, falling within the scope of the disclosure.

What is claimed is:

1. A suturing device, comprising:
a handle with a rod extending therefrom;
a needle member into which the rod is inserted to engage therewith; and
a suture attached to an outer surface of the needle member, wherein the needle member includes expandable fins formed at a proximate end thereof.

2. The suturing device of claim 1, wherein the needle member is hollow with a pointed tip at a distal end thereof to pierce through a tissue layer.

3. The suturing device of claim 1, wherein the needle member is a cylindrical hollow member with both ends thereof open.

4. The suturing device of claim 3, wherein the rod extending from the handle has a pointed tip that extends through the needle member when engaged therewith.

5. The suturing device of claim 2, wherein an interior of the needle member is magnetic to be engaged with a magnetic end of the rod.

6. The suturing device of claim 5, wherein the fins formed on the needle member expand to a diameter greater than an outer diameter of the needle member.

7. The suturing device of claim 6, wherein the fins retract to be in line with the outer surface of the needle member.

8. The suturing device of claim 6, wherein the fins expand after being inserted through the tissue layer to prevent backward movement into the tissue layer.

9. The suturing device of claim 1, wherein the needle member includes at least one protrusion formed along a circumferential surface thereof.

10. The suturing device of claim 1, wherein the needle member is expandable within a tissue layer.

11. The suturing device of claim 1, wherein a plurality of rods extend from the handle and a plurality of needle members are provided to receive each of the rods therein.

12. A suturing method, comprising:
engaging a rod extending from a handle of a suturing device into a needle member, wherein a suture is attached to an outer surface of the needle member;
piercing a tip of the needle member through a tissue layer, wherein fins formed at a proximate end of the needle member expand after the needle member passes through the tissue member.

13. The suturing method of claim 12, wherein as the needle member passes through the tissue layer, the fins are retracted to be in line with the outer surface of the needle member.

14. The suturing method of claim 12, wherein the fins expand to a diameter greater than an outer diameter of the needle member to prevent backward movement into the tissue layer.

15. The suturing method of claim 12, further comprising:
disengaging the rod from the needle member once the fins are expanded.

16. The suturing method of claim 15, further comprising:
reengaging the rod into the needle member to pass the needle member through another tissue layer.
